(19) United States
(12) Patent Application Publication
(10) Pub. No.: US 2024/0233908 A1
(43) Pub. Date: Jul. 11, 2024

(54) PROCESSING SYSTEM, PROCESSING METHOD, AND PROGRAM

(71) Applicant: TOYOTA JIDOSHA KABUSHIKI KAISHA, Toyota-shi (JP)

(72) Inventors: Issei NAKASHIMA, Katsushika-ku (JP); Toru MIYAGAWA, Seto-shi (JP); Hideo HASEGAWA, Nagoya-shi (JP); Yusuke KINOSHITA, Ota-ku (JP); Narimasa WATANABE, Chofu-shi (JP); Kohei OTSUKA, Sagamihara-shi (JP); Hiroki SATO, Chuo-ku (JP); Ryusuke KURODA, Edogawa-ku (JP)

(73) Assignee: TOYOTA JIDOSHA KABUSHIKI KAISHA, Toyota-shi (JP)

(21) Appl. No.: 18/404,323

(22) Filed: Jan. 4, 2024

(30) Foreign Application Priority Data
Jan. 5, 2023 (JP) 2023-000394

(51) Int. Cl. G16H 20/60; G16H 50/20

(52) U.S. Cl. CPC G16H 20/60; G16H 50/20

(57) ABSTRACT

A processing system according to an embodiment includes a meal data acquisition unit configured to acquire meal data related to meals consumed by a user, an exercise data acquisition unit configured to acquire exercise data related to exercise performed by the user, a prediction unit configured to generate prediction data predicting fluctuations in the user's blood sugar level based on the meal data and the exercise data, and a presentation unit configured to present a mealtime based on the prediction data.

### CROSS REFERENCE TO RELATED APPLICATIONS

This application is based upon and claims the benefit of priority from Japanese patent application No. 2022-000394, filed on Jan. 5, 2023, the disclosure of which is incorporated herein in its entirety by reference.

### BACKGROUND

The present disclosure relates to a processing system, a processing method, and a program.

Japanese Unexamined Patent Application Publication No. 2020-149326 discloses an apparatus that accepts input of events (such as meals and medical procedures including taking of medication) affecting blood sugar levels of a subject (patient) and then predicts the blood sugar level thereof. If the input event has an impact on the blood sugar level of the subject, the apparatus outputs alerts in the form of messages suggesting changes in the menu or quantity of the meal or alternative menu options.

### SUMMARY

In the above-mentioned apparatus, predicting a blood sugar level accurately is difficult, because it relies on predicting the blood sugar level based on a patient's meals and medical activities. The predictions may not be highly accurate. Moreover, the apparatus only suggests changes in the amount and menu of meals, which may not facilitate appropriate timings of meals. Consequently, there is a risk of significant fluctuations in the blood sugar level.

The present disclosure has been made to solve such a problem, and provides a processing system, a processing method, and a program capable of providing presentations to suppress fluctuations in a blood sugar level.

A processing system according to an embodiment includes a meal data acquisition unit configured to acquire meal data related to meals consumed by a user, an exercise data acquisition unit configured to acquire exercise data related to exercise performed by the user, a prediction unit configured to generate prediction data predicting fluctuations in the user's blood sugar level based on the meal data and the exercise data, and a presentation unit configured to present a mealtime based on the prediction data.

In the above processing system, the presentation unit may present a meal menu based on the prediction data.

In the above processing system, the presentation unit may present an exercise time and an exercise content based on the prediction data.

The above processing system may further include a schedule information acquisition unit configured to acquire schedule information indicating the user's meal schedule. The meal data in the future may be acquired based on the schedule information, and the prediction unit may predict the blood sugar level based on the meal data in the future.

In the above processing system, schedule information indicating the user's schedule may be acquired, and an additional plan to be added to free time in the schedule information may be presented based on the prediction data.

In the above processing system, schedule information indicating the user's schedule may be acquired, and the schedule may be adjusted based on the prediction data.

In the above processing system, user information about the user may be acquired, and the blood sugar level may be predicted using a personalized prediction model based on the user information.

A processing method according to the embodiment performed by at least one processor includes acquiring meal data related to meals consumed by a user, acquiring exercise data related to exercise performed by the user, generating prediction data predicting fluctuations in the user's blood sugar level based on the meal data and the exercise data, and presenting a mealtime based on the prediction data.

In the above processing method, a meal menu may be presented based on the prediction data.

In the above processing method, exercise times and exercise content may be presented based on the prediction data.

The above processing method may further include acquiring schedule information indicating the user's meal schedule. The meal data in the future may be acquired based on the schedule information, and the blood sugar level may be predicted based on the meal data in the future.

In the above processing method, schedule information indicating the user's schedule may be acquired, and an additional plan to be added to free time in the schedule information may be presented based on the prediction data.

In the above processing method, schedule information indicating the user's schedule may be acquired, and the schedule may be adjusted based on the prediction data.

In the above processing method, user information about the user may be acquired, and the blood sugar level may be predicted using a personalized prediction model based on the user information.

A program according to the embodiment causes a computer to execute acquiring meal data related to meals consumed by a user, acquiring exercise data related to exercise performed by the user, generating prediction data predicting fluctuations in the user's blood sugar level based on the meal data and the exercise data, and presenting a mealtime based on the prediction data.

According to the present disclosure, it is possible to provide a processing system, a processing method, and a program capable of providing presentations to suppress fluctuations in a blood sugar level.

The above and other objects, features and advantages of the present disclosure will become more fully understood from the detailed description given hereinbelow and the accompanying drawings which are given by way of illustration only, and thus are not to be considered as limiting the present disclosure.

### BRIEF DESCRIPTION OF DRAWINGS

FIG. 1 is a block diagram schematically showing a configuration of a processing system;

FIG. 2 is a flowchart for explaining a processing method according to an embodiment;

FIG. 3 is a graph showing predicted results of blood sugar levels by a prediction model; and

FIG. 4 is a graph showing predicted results of blood sugar levels by a prediction model.

### DESCRIPTION OF EMBODIMENTS

Hereinafter, the present disclosure will be described through an embodiment of the disclosure, but the claimed disclosure is not limited to the following embodiment. Furthermore, not all of the configurations described in the embodiment are essential as means for solving problems.

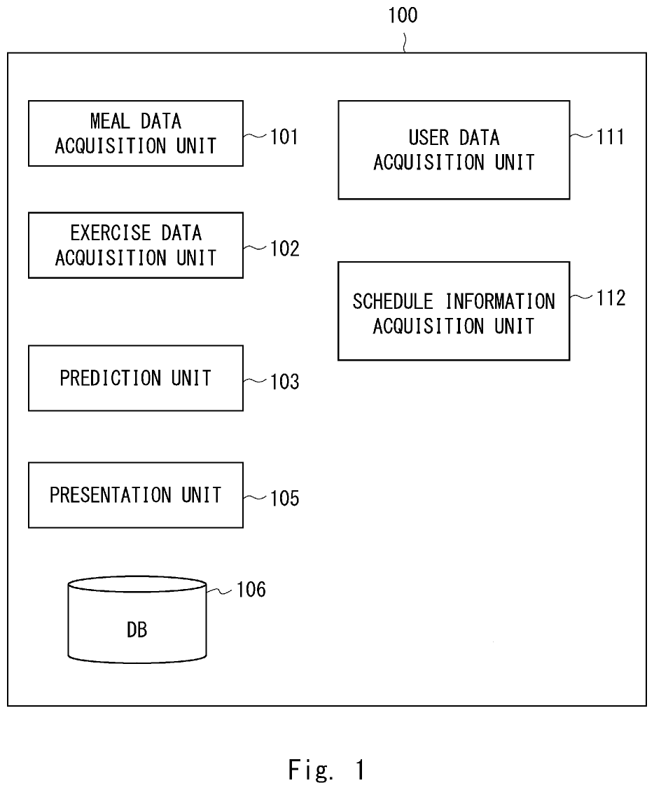

A processing system and a processing method according to the embodiment will be described with reference to the drawings. FIG. 1 is a block diagram showing a configuration of a processing system 100. The processing system 100 includes a meal data acquisition unit 101, an exercise data acquisition unit 102, a prediction unit 103, and a presentation unit 105. Further, the processing system 100 may include a user data acquisition unit 111 and a schedule information acquisition unit 112.

The processing system 100 may be implemented by one or more computers. The processing system 100 is typically an information processing apparatus such as a personal computer or a smartphone. Specifically, the processing system 100 includes a memory for storing programs and the like and a processor for executing the programs and the like. The processing system 100 may include a plurality of processors for performing distributed processing. For example, a server apparatus connected by a network and a user terminal may cooperate to perform processing.

The meal data acquisition unit 101 acquires meal data related to meals consumed by a user. The meal data acquisition unit 101 records the meal data in a memory or the like. The meal data indicates meal menus and mealtimes. The meal data acquisition unit 101 acquires data related to nutrients, components, ingredients, etc. from the meal menus consumed by the user. Preferably, the meal data includes a sugar content. The meal data includes not only breakfast, lunch, and dinner, but also snacks and late-night snacks.

For example, the meal data may include data related to the ingredients of the meal and their quantities in the meal. Alternatively, the meal data may include information such as calories, sugar content, salt content, fat content, vitamins, carbohydrates, and other nutrients. The meal data acquisition unit 101 acquires data on the consumed amount of each nutrient. In addition, the meal data includes data on mealtimes at which the user consumed the meals. The meal data acquisition unit 101 stores the time-series meal data in a database 106. The meal data includes the nutrients and meal menus associated with the meal data and mealtime.

The meal data can be acquired from image data captured of the user's meals. The meal data acquisition unit 101 may acquire the meal data using a sensor for detecting the meal data. The meal data acquisition unit 101 acquires the meal data from the detection result of the sensors. Alternatively, the user or other parties may input the consumed meal menus, so that the meal data acquisition unit 101 can acquire the meal data.

The exercise data acquisition unit 102 acquires the exercise data related to the exercise of the user. The exercise data indicates an exercise menu and an exercise time of the exercise performed by the user. Examples of the exercise include walking, jogging, cycling, foot-pedaling exercise using a foot-pedaling machine or the like. The exercise data may include data such as a heart rate and an exercise volume (amount of exercise). The exercise data may include data indicating exercise time including the start and end time of the exercise.

The user may input his/her performed exercise so that the exercise data acquisition unit 102 can acquire the exercise data. Alternatively, a wearable device such as a smartwatch may be used so that the exercise data acquisition unit 102 can detect the user's exercise. Further alternatively, a camera may be used to detect the user's exercise. In other words, the exercise data acquisition unit 102 may utilize a sensor to detect the exercise. The exercise data acquisition unit 102 acquires right brain data from the detection result of the sensor. The exercise data acquisition unit 102 records the time-series exercise data in the database 106 or the like. In the exercise data, the exercise volume and the exercise menu are associated with the exercise time.

The prediction unit 103 predicts the fluctuations in the user's blood sugar level based on the exercise data and the meal data and generates prediction data. The prediction unit 103 predicts the fluctuations in the user's blood sugar level. For example, the blood sugar level increases when the user has a meal. After a meal, the blood sugar level gradually decreases over time. In addition, engaging in exercise can suppress the increase in the blood sugar level or promote their reduction. The prediction unit 103 may use a machine learning model for predicting the fluctuations in the blood sugar level. That is, the prediction unit 103 may use a machine learning model using the meal data and the exercise data as input data. When new meal data and exercise data are input, the prediction unit 103 generates prediction data indicating future fluctuations in the blood sugar level.

The presentation unit 105 presents a mealtime to the user based on the prediction data. That is, the presentation unit 105 calculates an appropriate mealtime based on the prediction data and makes a recommendation. By doing so, the presentation unit 105 can provide presentations to help regulate fluctuations in the blood sugar level. The user can be informed of an appropriate timing to consume meals, enabling him/her to stay healthier.

For example, a recommended range of the blood sugar level (hereinafter referred to as a recommended blood sugar level range) is set in advance in the presentation unit 105. Based on the prediction result, the presentation unit 105 presents a recommended mealtime that helps keep the blood sugar level within the recommended blood sugar level range. The presentation unit 105 recommends the mealtime when the blood sugar level approaches a lower limit of the recommended blood sugar level range.

When the blood sugar level drops to an appropriate level, the user can have a meal. That is, it is possible to prevent the user from having a meal before the blood sugar level drops significantly. Alternatively, it is possible to prevent the user from having a meal when the blood sugar level is high. By spacing out meals so that there are appropriate intervals between them, rapid fluctuations in the blood sugar level can be suppressed. For example, it can help control the fluctuations in the blood sugar level so that they are within a preset allowable range.

The presentation unit 105 may also present a meal menu. For example, the presentation unit 105 may present a meal menu based on daily calories and nutrients that the user should consume. In other words, the presentation unit 105 compares the nutrients included in the already consumed meal with the nutrients that the user should consume and then presents a meal menu including the deficient nutrients. This way, the user can ensure that he/she consumes necessary nutrients and manages his/her blood sugar level more appropriately, leading to a healthier lifestyle. The presentation unit 105 may present the meal menu with the mealtime.

The user data acquisition unit 111 acquires data related to the user. For example, the user data acquisition unit 111 acquires data such as the user's height, weight, age, and gender. The user data may also include data related to the user's body fat percentage, body fat amount, and muscle mass. Further, the user data may also include data related to the user's occupation and medical history. The user data acquisition unit 111 records this user data in the database 106 or a memory.

The prediction unit 103 can perform predictions based on the user data. In other words, it is possible to achieve a prediction model that adjusts the likelihood of blood sugar levels decreasing or increasing based on the user data. By using a personalized prediction model tailored to user information, the prediction unit 103 can predict the blood sugar level of the user. This allows the prediction unit 103 to make more accurate predictions. Further, a daily recommended value and a daily recommended range of calories and nutrients that the user should consume may be defined based on the user data.

The schedule information acquisition unit 112 acquires schedule information of the user. The schedule information includes information indicating the user's schedule such as work, tasks, desk work, meetings, commuting, travel, meals, dining, snacks, exercise, kindergarten pick-up, and package deliveries. For each planned item, there are designated start and end times in the schedule information. The schedule information acquisition unit 112 records the schedule information in the database 106, a memory, or the like. The schedule information acquisition unit 112 can identify the user's free time based on the schedule information.

The presentation unit 105 may present the mealtime based on the schedule information. For example, the presentation unit 105 sets a mealtime during the user's free time. Specifically, the presentation unit 105 presents a mealtime excluding periods, for example, when the user is in a meeting or during travel. In other words, when there is a time slot during which the user cannot have a meal, the presentation unit 105 suggests having meals before or after the time slot.

Thus, the schedule information acquisition unit 112 can identify the user's free time based on the schedule information. Based on the prediction data, the presentation unit 105 presents the mealtime so that the user can have a meal during the user's free time. The presentation unit 105 presents a meal plan to the user as an additional plan to be added to the free time. In this way, a more appropriate presentation can be provided, leading to an improved state of the user's health.

Furthermore, when there is a meal schedule indicating a meal plan in the schedule information of the user, the prediction unit 103 may generate the prediction data based on the meal schedule. The meal schedule may include information indicating a mealtime, a meal location, a meal menu, etc. planned for the user. The planned mealtime may be set by the user, for example, or may be set from a restaurant reservation time, etc. If the user uses a company cafeteria or the like, the available time of the company cafeteria may be set as the planned mealtime. For example, the meal location, such as the company cafeteria, restaurant, or home, can be registered in the schedule.

The meal data based on the meal content to be consumed depending on the meal location is acquired. For each meal location, average or representative values of nutrients or the like to be consumed may be set in advance as the meal plan information. Alternatively, when a meal menu is reserved for dinner or the like, the meal plan information corresponding to the reservation is acquired. In this manner, the meal data acquisition unit 101 acquires future meal data (meal plan information) based on the schedule information. After that, the prediction unit 103 may make predictions based on the future meal data. For example, when a planned mealtime is set in the schedule information, the presentation unit 105 presents a time a certain period of time before the planned mealtime as a mealtime. This prevents the user from snacking right before the planned mealtime. In other words, the user can have an interval between mealtimes.

The prediction unit 103 may predict the fluctuations in the blood sugar level according to the planned item of the schedule. For example, the rate at which the blood sugar level decreases and the exercise volume may be set for each planned item. For example, in meetings or desk work, the rate of decrease in the blood sugar level and the exercise volume are low, while in exercise, walking, or cycling, the rate of decrease in the blood sugar level and the exercise volume are high. If the user's schedule involves physical labor, cycling, or walking, the processing system 100 may treat these planned items as exercise. That is, if there are planned items treated as exercise in the user's schedule, the exercise data acquisition unit 102 may acquire these planned items as future exercise data. The prediction unit 103 may predict the fluctuations in the blood sugar level based on these planned items.

The presentation unit 105 may present a meal menu based on the prediction data. The presentation unit 105 presents meal menus that help prevent a blood sugar level from becoming excessively high. Here, the meal menu may include information about the quantity of a meal to be consumed. In this way, it is possible to keep the blood sugar level within the recommended blood sugar level range. Therefore, the range of fluctuations in the blood sugar level can be suppressed, and the user can maintain a healthy lifestyle.

The presentation unit 105 may present the exercise time and the exercise content based on the prediction data. For example, if the blood sugar level exceeds an upper limit of the recommended blood sugar level range in the prediction data, the presentation unit 105 recommends the user to exercise. This can prevent a blood sugar level from rising too high. For example, the presentation unit 105 may present the exercise content (exercise menu) and the duration of the exercise.

The presentation unit 105 may refer to the schedule information when presenting the exercise. The exercise is presented during the time slots when the user can exercise. In other words, during the time slots when the user is unavailable for exercise, such as during a meeting, the presentation unit 105 does not present the exercise.

The schedule information acquisition unit 112 refers to the schedule information to identify free time (spare time). The time slots in the schedule where no appointments are registered are considered as free time. The presentation unit 105 presents exercises during the free time based on the prediction data. In other words, the presentation unit 105 presents exercise as an additional planned item to be added to the free time. This way, the processing system 100 can present more appropriate presentations and effectively control the increase in a blood sugar level, leading to an improved state of the user's health.

In this way, the prediction unit 103 predicts the fluctuations in the blood sugar level. Therefore, it can estimate the time slots or times that are not suitable for meals. In other words, the presentation unit 105 presents the mealtime to ensure that a blood sugar level stays within the recommended blood sugar level range. The user may be notified with an alert if snacking occurs during times that are not suitable for meals to restrict snacking. This helps prevent a blood sugar level from exceeding the upper limit of the recommended blood sugar level range. Additionally, the presentation unit 105 can refer to the schedule information to estimate the time slots or times that are suitable for meals. This allows the user to lead a healthier lifestyle.

Furthermore, when a suitable time for meals is estimated, the presentation unit 105 may recommend a schedule change to the user according to the estimated time. For example, the presentation unit 105 may suggest a rescheduling of a meeting time or a cancellation of the meeting. When a time that the user should exercise is estimated, a schedule change may be presented so that the exercise can be performed. A schedule change may be presented by the presentation unit 105 so that the user can exercise or work at an appropriate time. For example, the presentation unit 105 may adjust delivery time of a package, meeting times, kindergarten pickup times, and the like.

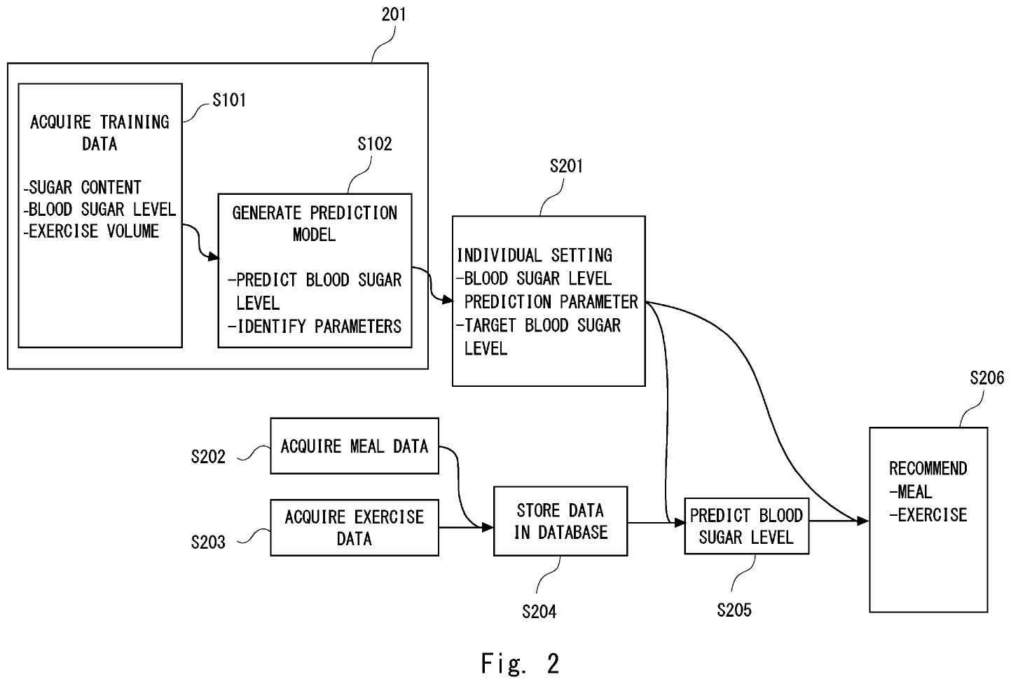

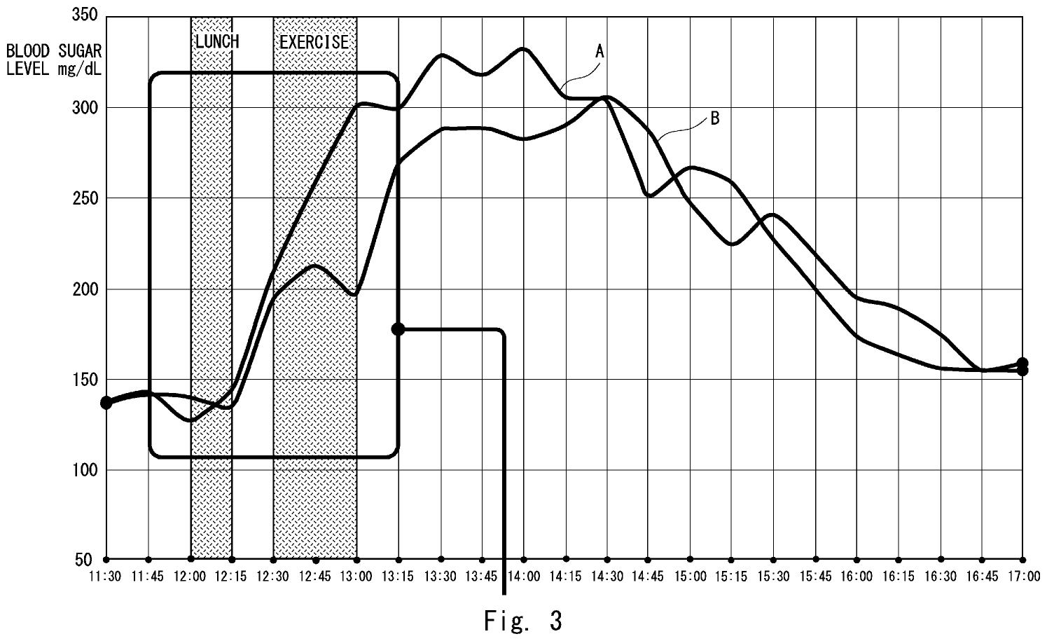

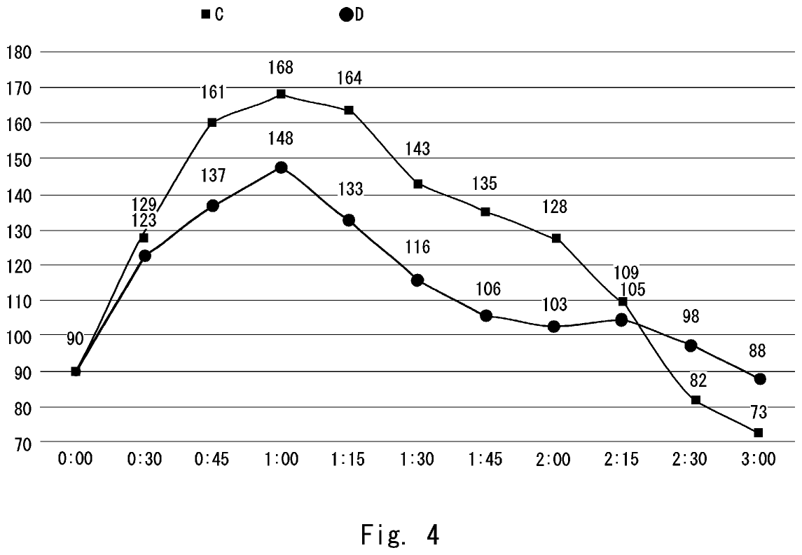

Next, the generation and use of a prediction model for predicting the fluctuations in the blood sugar level will be described. FIG. 2 is a schematic diagram showing the generation and use flow of the prediction model. FIGS. 3 and 4 show graphs of the prediction model for predicting the fluctuations in the blood sugar level.

First, the generation of the prediction model will be described. Here, a learning unit 201 performs machine learning to generate a prediction model. First, the learning unit 201 acquires training data for generating the prediction model (S101). The training data includes data such as a sugar content consumed by the user, the user's blood sugar level, and the exercise volume. The training data includes measurement data of the blood sugar level.

The measurement data of the blood sugar level is acquired, for example, as time-series data. The user wears a sensor for monitoring the blood sugar level. This allows for the blood sugar level during meals or exercise to be acquired. In other words, when the user wears a blood glucose monitor (a blood sugar level monitor), measured values of the blood sugar level during meals or exercise can be acquired. The time-series data of the sugar content and the exercise volume may be input by the user or detected by the sensor.

Next, a prediction model is generated through machine learning using the blood sugar levels, the sugar content, and the exercise volume as the training data (S102). The prediction model is generated by supervised learning, where the measurement data of the blood sugar level is used as a ground truth label (also referred to as a teacher signal). The prediction model is a machine learning model generated through supervised learning. The machine learning model uses the sugar content and the exercise volume as input data, and the blood sugar level as output data.

The learning unit 201 identifies parameters for predicting the blood sugar level. The learning unit 201 may construct a learning model through deep learning. The learning unit 201 may construct a learning model using a CNN (Convolutional Neural Network) that performs convolution operations. In this case, the prediction model may have a convolution layer, a pooling layer, or the like. The learning unit 201 may construct a learning model using an RNN (Recurrent Neural Network) that handles time-series data. For example, the learning unit 201 may use time-series data such as the blood sugar level, the exercise volume, and sugar content as the training data.

The learning unit 201 updates parameters such as weights of the neural network based on the ground truth label. That is, the learning unit 201 calculates parameters that minimize the error function between the prediction and the ground truth label. The measurement data of the blood sugar level is used as the ground truth label. When a multidimensional vector including the exercise data and the meal data is input to the prediction model, the blood sugar level is output. Furthermore, by sequentially inputting the time-series data, the prediction model can predict the fluctuations in the blood sugar level.

The neural network can be used as an algorithm, regardless of the type of prediction model or its algorithm to be trained by the learning unit 201. In particular, it is preferable to use a deep neural network (DNN) that has multiple intermediate layers. For example, a feed-forward neural network such as a multilayer perceptron (MLP) that employs the error backpropagation method may be used as the DNN.

Further, the learning unit 201 may perform machine learning using user data. For example, the user data is data indicating the user's weight, height, gender, and age. The learning unit 201 can construct a prediction model using the user data as input. This allows for generation of a more accurate prediction model. For example, the learning unit 201 can perform machine learning based on the user data such as the user's weight, height, and gender. Additionally, it becomes possible to generate a prediction model without directly measuring the actual blood sugar level of the user.

The training data can also use actual measurement data of the user for predicting the blood sugar level. Alternatively, the training data may include measurement data from a plurality of users. Using the measurement data from the plurality of users allows for preparation of a large amount of training data. The learning unit 201 can personalize the prediction model by performing machine learning using the user data. In this case, the learning unit 201 can perform the machine learning using the user data as the input data. Therefore, the input data for the prediction model is a multidimensional vector that includes the meal data, the exercise data, and the user data. It is possible to generate a prediction model that predicts a blood sugar level tailored to the user. The learning unit 201 can generate a personalized prediction model. This allows for generation of a prediction model with high prediction accuracy.

Next, the utilization stage of the prediction model is described. The user or the processing system 100 performs individual settings (S201). First, the parameters obtained in Step S102 are set in the prediction model. The user or the processing system 100 sets a target blood sugar level. The target blood sugar level may be a recommended blood sugar level range defined by upper and lower limits. Alternatively, the target blood sugar level may only indicate the upper limit. The processing system 100 may automatically set the target blood sugar level based on the user data such as the user's height, age, weight, and gender.

Next, the meal data acquisition unit 101 acquires the user's meal data (S202). The exercise data acquisition unit 102 acquires the exercise data (S203). The meal data and the exercise data may be data detected by a sensor or data input by the user by operating a touch panel or the like. The meal data acquisition unit 101 and the exercise data acquisition unit 102 store the meal data and the exercise data as time-series data in the database 106 (S204).

Next, the processing system 100 inputs the meal data and the exercise data stored in the database 106 to the prediction model to predict the blood sugar level (S205). By inputting the time-series meal data and exercise data to the prediction model, the processing system 100 estimates the fluctuations in the blood sugar level. The prediction model uses the meal data and the exercise data to generate the prediction data for the blood sugar level. The prediction data represents the time-series data indicating the fluctuations in the blood sugar level. Obviously, the prediction model may also be a machine learning model that takes user data as the input data.

Next, the presentation unit 105 recommends meals or exercises based on the prediction data (S206). For example, the presentation unit 105 recommends mealtimes that do not cause the blood sugar level to exceed the recommended blood sugar level range. Furthermore, the presentation unit 105 may present meal menus, exercise time, and exercise menus that do not cause the blood sugar level to exceed the recommended blood sugar level range. By doing so, the user can engage in exercise and meals at optimal times, effectively managing a blood sugar level.

The presentation unit 105 may refer to the schedule information to present mealtimes or the like. By presenting meals or exercises during the free time in the schedule, effective recommendations can be made.

The schedule information may also include meal schedules. In other words, the schedule information acquisition unit 112 acquires the schedule information indicating meal schedules.

If there is a meal schedule indicating a future meal plan in the schedule information, the meal data acquisition unit 101 may acquire the future meal plan. In this case, the meal data including the future meal plan is sequentially input to the prediction model. The prediction unit 103 predicts a blood sugar level based on the future meal data. The presentation unit 105 presents meals and exercise using the future meal data.

If the mealtime presented to the user overlaps with the planned time in the user's schedule, the presentation unit 105 may present a schedule change to the user. This allows the user to engage in meals and exercises at appropriate times. Since the user can engage in meals and exercises at optimal times, he/she can manage the blood sugar level effectively.

FIG. 3 is a graph showing a result of predicting the blood sugar level using the prediction model. The horizontal axis represents time, and the vertical axis represents the blood sugar level in [mg/dl]. FIG. 3 shows the fluctuations in the blood sugar level after the user had lunch from 12:00 to 12:15. The graph also displays the predicted results, labeled as Result A, when the user remained at rest after lunch, and Result B, when the user engaged in walking from 12:30 to 13:00. As the meal is digested, the blood sugar level gradually increases. By engaging in exercise after a meal, the increase in a blood sugar level can be suppressed.

FIG. 4 is a graph showing the results of predicting a blood sugar level using another prediction model. The horizontal axis represents time, and the vertical axis represents the blood sugar level. The graph shows the fluctuations in the blood sugar level after the user had a meal. In this case, the meal consists of 100 g of white rice. The graph also shows the predicted result for when no exercise is performed after the meal (Result C) and when cycling exercise is performed for 30 minutes after the meal (Result D). As white rice is digested, the blood sugar level increases. By engaging in exercise after a meal, the increase in a blood sugar level can be suppressed. Furthermore, exercise can promote a decrease in a blood sugar level.

In both cases, the prediction models can accurately predict the fluctuations in a blood sugar level. It is possible to model the increase in a blood sugar level due to meals and the suppression of a blood sugar level increase due to exercise.

The processing system 100 and the learning unit 201 are not limited to being physically confined to a single apparatus. For example, the prediction model generated by the learning unit 201 through machine learning may be stored on a server different from the user terminal. For example, the meal data and the exercise data acquired by the user terminal are transmitted to the server. The server, which stores the prediction models, generates the prediction data and transmits it back to the user terminal. Then, the user terminal presents meals and exercise.

Some or all of the above processing may be executed by a computer program. That is, a control computer constituting the processing system 100 executes the program to control the processing system 100.

The program can be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g. magneto-optical disks), CD-ROM (compact disc read only memory), CD-R (compact disc recordable), CD-R/W (compact disc rewritable), and semiconductor memories (such as mask ROM, PROM (programmable ROM), EPROM (erasable PROM), flash ROM, RAM (random access memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g. electric wires, and optical fibers) or a wireless communication line.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A processing system comprising:
a meal data acquisition unit configured to acquire meal data related to meals consumed by a user;
an exercise data acquisition unit configured to acquire exercise data related to exercise performed by the user;
a prediction unit configured to generate prediction data predicting fluctuations in the user's blood sugar level based on the meal data and the exercise data; and
a presentation unit configured to present a mealtime based on the prediction data.

2. The processing system according to claim 1, wherein the presentation unit presents a meal menu based on the prediction data.

3. The processing system according to claim 1, wherein the presentation unit presents an exercise time and an exercise content based on the prediction data.

4. The processing system according to claim 1, further comprising a schedule information acquisition unit configured to acquire schedule information indicating the user's meal schedule, wherein
the meal data in the future is acquired based on the schedule information, and
the prediction unit predicts the blood sugar level based on the meal data in the future.

5. The processing system according to claim 1, wherein
schedule information indicating the user's schedule is acquired, and
an additional plan to be added to free time in the schedule information is presented based on the prediction data.

6. The processing system according to claim 1, wherein
schedule information indicating the user's schedule is acquired, and
the schedule is adjusted based on the prediction data.

7. The processing system according to claim 1, wherein
user information about the user is acquired, and
the blood sugar level is predicted using a personalized prediction model based on the user information.

8. A processing method performed by at least one processor, the processing method comprising:
acquiring meal data related to meals consumed by a user;
acquiring exercise data related to exercise performed by the user;
generating prediction data predicting fluctuations in the user's blood sugar level based on the meal data and the exercise data; and
presenting a mealtime based on the prediction data.

9. The processing method according to claim 8, wherein the meal menu is presented based on the prediction data.

10. The processing method according to claim 8, wherein the exercise time and the exercise content are presented based on the prediction data.

11. The processing method according to claim 8, further comprising acquiring schedule information indicating the user's meal schedule, wherein
the meal data in the future is acquired based on the schedule information, and
the blood sugar level is predicted based on the meal data in the future.

12. The processing method according to claim 8, wherein
schedule information indicating the user's schedule is acquired, and
an additional plan to be added to free time in the schedule information is presented based on the prediction data.

13. The processing method according to claim 8, wherein
schedule information indicating the user's schedule is acquired, and
the schedule is adjusted based on the prediction data.

14. The processing method according to claim 8, wherein
user information about the user is acquired, and
the blood sugar level is predicted using a personalized prediction model based on the user information.

15. A non-transitory computer readable medium storing a program for causing a computer to execute:
acquiring meal data related to meals consumed by a user;
acquiring exercise data related to exercise performed by the user;
generating prediction data predicting fluctuations in the user's blood sugar level based on the meal data and the exercise data; and
presenting a mealtime based on the prediction data.
